(19) United States
(12) Patent Application Publication
(10) Pub. No.: US 2021/0318323 A1
(43) Pub. Date: Oct. 14, 2021

(54) Information Acquiring Method, Information Acquiring Device, and Program

(71) Applicant: KONICA MINOLTA INC., Chiyoda-ku (JP)

(72) Inventors: Kenji NISHIKAWA, Hino-shi (JP); Masaru TAKAHASHI, Kokubunji-shi (JP)

(21) Appl. No.: 17/273,262

(22) PCT Filed: Sep. 5, 2019
(86) PCT No.: PCT/JP2019/035059

§ 371 Date: Mar. 3, 2021

(30) Foreign Application Priority Data
Sep. 6, 2018 (JP) 2018-166518

(51) Int. Cl. G01N 33/574; G01N 1/30

(52) U.S. Cl. CPC G01N 33/57492; G01N 1/30

(57) ABSTRACT

Information useful for diagnosis or treatment is acquired by quantitatively evaluating the expression levels of each of a plurality of mutually different domains in the same protein in a human tissue slice, by providing: a staining process of using a plurality of staining reagents having mutually different colorings to stain the plurality of different domains in the same protein; a quantifying step of quantifying the expression levels of each of the plurality of domains; and a calculating step of calculating the ratios between the expression levels of the plurality of domains.

### TECHNOLOGICAL FIELD

The present invention relates to an information acquiring method, an information acquiring device, and a program.

### BACKGROUND ART

In pathological diagnosis, quantification of an expression level of a biological material overexpressing in a tissue section provides very important information for predicting a prognosis of a patient and for deciding a treatment plan afterward. In particular, quantitative assessment of an expression level of oncoprotein in cells provides a vital clue in determining malignancy of cancer.

Conventionally, a technique of fluorescent labeling using phosphor integrated dots to know an expression status of a target biological material has been known (see, for example, Patent Document 1). Phosphor integrated dots are bound with a biological material recognition site that can recognize and bind with a target biological material. Specifically, a tissue specimen is stained with the phosphor integrated dots. The peak of brightness distribution of fluorescent bright spots is analyzed to obtain an average brightness value of one particle. The number of particles within each bright spot is calculated. An expression level of a target biological material is evaluated by comparing the calculated number of particles. Since a brightness value of one particle of the phosphor integrated dots is high, a small amount of a biological material is quantitatively detected.

For example, in recent years, anti-PD-L1 antibody targeted on a PD-1/PD-L1 immune checkpoint has been developed. It is hopeful molecular targeted therapeutic medicine for malignant melanoma, lung cancer, etc. PD-L1 is a transmembrane protein specifically expressed in cancerous cells and has intracellular or extracellular domains. An anti-PD-L1 antibody that recognizes these domains has been developed. A fluorescent substance is bound with such an antibody. A biological material corresponding to a biological material recognition site of the antibody is labeled with fluorescence. Thereby an expression status of PD-L1, which is a target biological material, is known.

It is generally known that domains on a protein have genetically different developmental mechanisms and have independent functions respectively. Therefore, comparison of expression statuses of different domains on one protein and analysis of their relation may provide information about various proteins including PD-L1 which can be used in treatment and diagnosis. However, conventional diagnoses have been based on staining of a single domain.

### PRIOR ART DOCUMENT

### Patent Document

Patent Document 1: WO 2012/029342 A

### SUMMARY OF THE INVENTION

### Problems to be Solved by the Invention

It is an object of the present invention to provide an information acquiring method, an information acquiring device and a program that acquires information useful for diagnosis or treatment by quantitatively evaluating an expression level of each of domains in one protein.

### Means for Solving Problems

In order to solve the above problems, the inventors considered methods of separately staining intracellular domains and extracellular domains of one protein. As a result, the inventors succeeded in clearly distinguishing and observing domains by staining the domains with staining reagents of different colors. In addition, the inventors quantitatively assessed expression levels of domains using samples from cancer patients and found a correlation between prognoses of patients and expression levels of domains. Thus, the present invention relating to an information acquiring method utilizing a quantitative relation of domains has been achieved.

The present invention solves the problem by the following means.

According to an first aspect of the invention, an information acquiring method includes:

staining different domains of one protein in a tissue section of a human using staining reagents of different colors;

quantifying an expression level of each of the domains; and

calculating a ratio between expression levels of the domains.

A second aspect of the invention is the information acquiring method according to claim 1, wherein the tissue section is a sample from a tumor tissue of the human

A third aspect of the invention is the information acquiring method according to claim 1 or 2, wherein the staining of the domains is performed in an immunohistochemical staining method.

A fourth aspect of the invention is the information acquiring method according to claim 3, wherein

each of the staining reagents is a combination of a biological material recognition site and phosphor integrated dots integrated with fluorescent substances;

the quantifying includes counting a number of bright spots of the phosphor integrated dots; and

in the calculating, a ratio between numbers of bright spots is calculated, the numbers being counted for the domains.

A fifth aspect of the invention is the information acquiring method according to any one of claims 1 to 4, further including:

generating evaluation support information based on the ratio between the expression levels to predict prognosis of the human who provided the tissue section.

According to a sixth aspect of the invention, an information acquiring device acquires information from a tissue section of a human by staining different domains of one protein in the tissue section using staining reagents of different colors, the device including:

a quantification unit that quantifies an expression level of each of the domains; and

a calculator that calculates a ratio between expression levels of the domains.

A seventh aspect of the invention is a program for a computer of an information acquiring device that acquires information from a tissue section of a human by staining different domains of one protein in the tissue section using staining reagents of different colors, the program making the computer function as:

a quantification unit that quantifies an expression level of each of the domains; and

a calculator that calculates a ratio between expression levels of the domains.

### Advantageous Effects of Invention

The present invention provides an information acquiring method, an information acquiring device and a program that acquires information useful for diagnosis or treatment by quantitatively evaluating an expression level of each of domains in one protein.

### BRIEF DESCRIPTION OF THE DRAWINGS

FIG. 1 shows an outline of configuration of a pathological diagnosis support system according to the present invention.

FIG. 2 is a block diagram showing a functional configuration of an information acquiring device in FIG. 1.

FIG. 3 shows a relation between expression levels of domains and survival periods of patients.

FIG. 4 shows a relation between the expression levels of domains and survival rates of patients.

### EMBODIMENTS FOR CARRYING OUT THE INVENTION

Hereinafter, an embodiment for carrying out the present invention will be described, but the present invention is not limited thereto.

Configuration of Pathological Diagnosis Support System 100

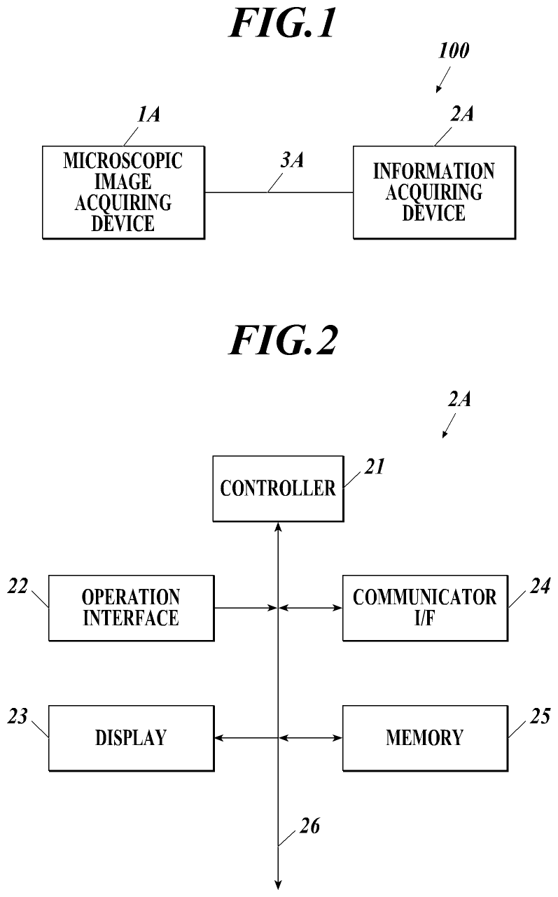

FIG. 1 shows an overall configuration example of a pathological diagnosis support system 100 that performs an information acquiring method of the invention. The pathological diagnosis support system 100 acquires a microscopic image of a tissue specimen stained with predetermined staining reagents and analyzes the acquired microscopic image. The system quantitatively outputs a feature amount that represents an amount of a specific biological material appearing in the tissue specimen of an observation target.

As shown in FIG. 1, the pathological diagnosis support system 100 is configured such that the microscopic image acquiring device 1A and the information acquiring device 2A are connected so as to be able to transmit and receive data via an interface, such as a cable 3A. The connection between the microscope image acquiring device 1A and the information acquiring device 2A is not particularly limited. For example, the microscope image acquiring device 1A and the information acquiring device 2A may be connected via a LAN (Local Area Network) or may be connected wirelessly.

The microscopic image acquiring device 1A is a well-known optical microscope with a camera which obtains the microscopic image of the tissue specimen on a slide placed on a slide fixing stage and sends it to the information acquiring device 2A.

The microscopic image acquiring device 1A includes an irradiating unit, an image forming unit, an imaging unit, a communicator I/F, and the like. The irradiating unit includes a light source, a filter, and the like, and irradiates the tissue specimen on the slide placed on the slide fixing stage with light. The image forming unit includes an ocular lens, an object lens, and the like, and forms an image of transmitted light, reflected light, or fluorescence from the tissue specimen on the slide due to the irradiated light. The imaging unit is a camera provided in a microscope which includes a CCD (Charge Coupled Device) sensor, and the like, and captures an image on an image forming face formed by the image forming unit to generate digital image data of the microscopic image. The communicator I/F transmits the image data of the generated microscopic image to the information acquiring device 2A. In this embodiment, the microscopic image acquiring device 1A includes a bright field unit which is combination of the irradiating unit and the image forming unit suitable for bright field observation and a fluorescent unit which is combination of the irradiating unit and the image forming unit suitable for fluorescence observation. The bright field and fluorescence are switched by switching the units.

The microscopic image acquiring device 1A is not limited to a microscope having a camera. For example, a virtual microscope slide creating device which scans a slide on a slide fixing stage of a microscope and obtains a microscopic image of the entire tissue specimen may be used (for example, see Japanese Patent Application Laid-Open Publication No. 2002-514319). According to the virtual microscope slide creating device, image data can be obtained with which the entire image of the tissue specimen on the slide can be viewed at once on a display.

The information acquiring device 2A analyzes the microscopic image transmitted from the microscopic image acquiring device 1A to calculate distribution of specific biological materials appearing in the tissue specimen of the observation target.

FIG. 2 shows an example of a functional configuration of the information acquiring device 2A. As shown in FIG. 2, the information acquiring device 2A includes a controller 21, an operation interface 22, a display 23, a communicator I/F 24, a memory 25, and the like. These components are connected through a bus 26.

The controller 21 includes a CPU (Central Processing Unit), a RAM (Random Access Memory), and the like, performs various processing in coordination with various programs stored in the memory 25, and collectively controls operation of the information acquiring device 2A. For example, the controller 21 functions as a quantification unit that performs quantification of an expression level of a domain and as a calculator that performs calculation to obtain a ratio between expression levels of domains in coordination with programs stored in the memory 25.

The operation interface 22 includes a keyboard provided with character input keys, numeric input keys, and various function keys and a pointing device such as a mouse, and outputs depression signals of the pressed keys of the keyboard and operation signals of the mouse as the input signal to the controller 21.

The display 23 includes, for example, a monitor such as a CRT (Cathode Ray Tube), an LCD (Liquid Crystal Display), and the like, and displays various screens according to an instruction of a display signal input from the controller 21.

The communicator I/F 24 is an interface for transmitting and receiving data to and from external devices such as the microscopic image acquiring device 1A. The communicator I/F 24 functions as a means for inputting a fluorescence image taken by the microscopic image acquiring device 1A to the information acquiring device 2A.

The memory 25 includes, for example, an HDD (Hard Disk Drive), a nonvolatile semiconductor memory, and the like. The memory 25 stores various programs and various pieces of data as described above.

Other than the above, the information acquiring device 2A may include a LAN adaptor, a router, and the like, and may be connected to external devices through a communication network such as a LAN.

Information Acquiring Method

The information acquiring method of the present invention will be described below.

The information acquiring method according to a preferred embodiment of the present invention is a method for specifically detecting biological materials from pathological sections.

The method essentially includes:

(i) Step of staining pathological sections using staining reagents;

(ii) Step of detecting domains of biological materials from pathological sections after staining; and

(iii) Step of assessing expression levels of domains of detected biological materials.

In particular, in Step (i), two kinds of fluorescent nanoparticles are used as staining reagents to stain different domains in one protein (biological material).

Step (iii) includes:

a step of quantifying expression levels of domains;

a step of calculating a ratio between expression levels of domains; and

a step of generating evaluation support information based on the ratio between the expression levels of domains.

Fluorescent nanoparticles are nanosized particles that fluoresces in response to excitation light and emits fluorescence that is intense enough to express each molecule of a target biological material as a bright spot.

Preferably, phosphor integrated dots (PID) are used as the fluorescent nanoparticles.

A first nanoparticle is bound with a biological material recognition site that recognizes a first domain and contains a predetermined fluorescent substance. A second nanoparticle is bound with a biological material recognition site that recognizes a second domain which is different from the first domain recognized by the biological material recognition site of the first nanoparticle, and contains a fluorescent substance with a fluorescent wavelength different from that of the fluorescent substance of the first nanoparticle.

Thus, nanoparticles are bound with different biological material recognition sites, and contains fluorescent substances with different fluorescent wavelengths. Since the fluorescent wavelengths of the fluorescent substances are different, different domains in one protein are clearly distinguished and are separately stained.

In the preferred embodiment of the present invention, an example using two kinds of nanoparticles are shown. However, as long as biological material recognition sites and fluorescent substances (fluorescent wavelengths) are different from each other, three or more kinds of domains can be detected with three or more kinds of nanoparticles.

Kinds and properties of the fluorescent substances, and details of a method for detecting biological materials will be described below.

(1) Target Biological Material

A target biological material is a subject of immunohistochemical staining with a fluorescent label, mainly for detecting or quantifying in terms of pathological diagnostics. The subject is a biological material expressed in a tissue section, especially a protein (antigen).

A target biological material that can be applied to the embodiment is the one which is expressed in a cell membrane of various tumor tissues, which is utilized as a biomarker, and which has intracellular domains and extracellular domains.

Examples of such target biological material are PD-L1, HER2 and TIM-3, but the target biological material is not limited to these.

(2) Phosphor Integrated Dots

The phosphor integrated dot is a nanosized particle having a particle of organic or inorganic substance as a base. Fluorescent substances (e.g., a fluorescent organic dye or a quantum dot, which will be described later) are contained in the base and/or adsorbed on the surface of the base.

Preferably, the base of the phosphor integrated dot and the fluorescent substances have substituents or sites with charges opposite to each other to cause electrostatic interactions.

A fluorescent dye integrated dot, a quantum dot integrated dot, etc. are used as the phosphor integrated dot.

In the embodiment, two kinds of primary antibodies that recognize different domains of one protein (biological material) are used, as described below. Also, two kinds of phosphor integrated dots that respectively bind to the primary antibodies are used. It is desirable that a difference in the maximum wavelength of fluorescence between the two kinds of phosphor integrated dots is 50 nm or more.

(2.1) Fluorescent Substance

Examples of the fluorescent substance used in the staining reagent for obtaining the fluorescence image include a fluorescent organic dye and a quantum dot (semiconductor particles). Preferably, the substance exhibits emission of visible to near infrared rays having a wavelength within the range from 400 to 1000 nm when excited by ultraviolet to near infrared rays having a wavelength within the range from 200 to 700 nm.

Examples of the fluorescent organic dye include fluorescein dye molecules, rhodamine dye molecules, Alexa Fluor (manufactured by Invitrogen Corporation) dye molecules, BODIPY (manufactured by Invitrogen Corporation) dye molecules, cascade dye molecules, coumarin dye molecules, eosin dye molecules, NBD dye molecules, pyrene dye molecules, Texas Red dye molecules and cyanine dye molecules.

Specific examples thereof include 5-carboxy-fluorescein, 6-carboxy-fluorescein, 5,6-dicarboxy-fluorescein, 6-carboxy-2′,4,4′,5′,7,7′-hexachlorofluorescein, 6-carboxy-2′,4,7,7′-tetrachlorofluorescein, 6-carboxy-4′,5′-dichloro-2′,7′-dimethoxyfluorescein, naphthofluorescein, 5-carboxy-rhodamine, 6-carboxy-rhodamine, 5,6-dicarboxy-rhodamine, rhodamine 6G, tetramethylrhodamine, X-rhodamine, and Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Al′xa Fluor 568, Alexa Fluor 594, Alexa Fluor 610, Alexa Fluor 633, Alexa Fluor 635, Alexa Fluor 647, Alexa Fluor 660, Alexa Fluor 680, Alexa Fluor 700, Alexa Fluor 750, BODIPY FL, BODIPY TMR, BODIPY 493/503, BODIPY 530/550, BODIPY 558/568, BODIPY 564/570, BODIPY 576/589, BODIPY 581/591, BODIPY 630/650, BODIPY 650/665 (the above are manufactured by Invitrogen Corporation), methoxycoumalin, eosin, NBD, pyrene, Cy5, Cy5.5 and Cy7. These can be used individually, or be used by mixing several kinds thereof.

Usable examples of the quantum dot include quantum dots respectively containing, as a component, II-VI compounds, III-V compounds, and IV elements (called “II-VI quantum dot”, “III-V quantum dot” and “IV quantum dot”, respectively). These can be used individually, or be used by mixing several kinds thereof.

Specific examples thereof include but are not limited to CdSe, CdS, CdTe, ZnSe, ZnS, ZnTe, InP, InN, InAs, InGaP, GaP, GaAs, Si and Ge.

A quantum dot having a core of any of the above quantum dots and a shell provided thereon can also be used. Hereinafter, as a notation for the quantum dot having a shell, when the core is CdSe and the shell is ZnS, the quantum dot is noted as CdSe/ZnS. Usable examples of the quantum dot include but are not limited to CdSe/ZnS, CdS/ZnS, InP/ZnS, InGaP/ZnS, Si/SiO2, Si/ZnS, Ge/GeO2, and Ge/ZnS.

A quantum dot surface-treated with an organic polymer or the like may be used as needed. Examples thereof include CdSe/ZnS having a surface carboxy group (manufactured by Invitrogen Corporation) and CdSe/ZnS having a surface amino group (manufactured by Invitrogen Corporation).

In addition to the fluorescent organic dye and the quantum dots, a “long afterglow phosphor” may be used as a fluorescent substance to be integrated into the phosphor integrated dots. The long afterglow phosphor includes Y2O3, Zn2SiO4, etc. as a base. Mn2+, Eu3+, etc. are used as an activator.

(2.2) Base

Examples of an organic substance used for the base include:

resin generally classified as thermosetting resin, such as melamine resin, urea resin, aniline resin, guanamine resin, phenolic resin, xylene resin, and furan resin;

resin generally classified as thermoplastic resin, such as styrene resin, acrylic resin, acrylonitrile resin, AS resin (acrylonitrile-styrene copolymer), and ASA resin (acrylonitrile-styrene-methyl acrylates copolymer);

other resin such as polylactic acid; and

polysaccharide.

Examples of an inorganic substance used for the base are silica, glass, etc.

(2.3) Quantum Dot Integrated Dots

The quantum dot integrated dots have configuration in which quantum dots are contained in the base and/or adsorbed on the surface of the base.

In a case in which quantum dots are contained in the base, the quantum dots may or may not be chemically bound with the base itself as long as the quantum dots are dispersed in the base.

(2.4) Fluorescent Dye Integrated Dots

The fluorescent dye integrated dots have configuration in which the fluorescent organic dye is contained in the base and/or adsorbed on the surface of the base.

In a case in which the fluorescent organic dye is contained in the base, the fluorescent organic dye may or may not be chemically bound with the base itself as long as the fluorescent organic dye may be dispersed in the base.

(2.5) Preparation of Phosphor Integrated Dots

The phosphor integrated dots can be made in common methods.

Specifically, to make, for example, silica particles including a base of silica and a fluorescent substance in the base, the fluorescent substances, such as the quantum dots and the fluorescent organic dye, and a silica precursor, such as tetraethoxysilane, are dissolved in a solution. The solution is added dropwise to a solution in which ethanol and ammonia are dissolved, and the silica precursor is hydrolyzed.

To make the phosphor integrated resin dots including a base of resin and a fluorescent substance contained in a resin particle or adsorbed on the surface of a resin particle, a solution of the resin or a dispersion of fine particles is prepared in advance. The fluorescent substances, such as the quantum dots and the fluorescent organic dye, are added to the dispersion and are stirred. Thereby the phosphor integrated resin dots are prepared. Alternatively, the phosphor integrated resin dots may be prepared by adding the fluorescent substances to a solution of a resin material and then causing polymerization reaction.

For example, in a case in which thermosetting resin such as melamine resin is used for the base, a reaction mixture containing a resin material (monomer, oligomer or prepolymer, e.g., methylol melamine, which is a condensation product of melamine and formaldehyde) and the fluorescent organic dye is heated. Polymerization reaction is caused in the emulsion polymerization method. Thus, the phosphor integrated resin dots are prepared. Preferably, the reaction mixture further includes a surfactant and a polymerization reaction accelerator such as acid. In a case in which thermoplastic resin such as a styrene-based copolymer is used for the base, a reaction mixture including a resin material, the fluorescent organic dye and a polymerization initiator (benzoyl peroxide, azobisisobutyronitrile, etc.) is heated. As a material monomer for the resin, a monomer with which the organic fluorescent dye is bound in advance by a covalent bond or the like may be used. Polymerization reaction is caused in the radical polymerization method or in the ionic polymerization method. Thus, the phosphor integrated resin dots are prepared.

(2.6) Average Particle Size

The average particle size of the phosphor integrated dots used in the embodiment is not limited. Those with large particle size have less access to antigen. Those with small particle size have a lower brightness value so that signals of the phosphor integrated dots are buried in background noise (camera noise or auto fluorescence of cells). Therefore, those of about 20-500 nm are suitable.

A coefficient of variation (=(standard deviation/mean)×100%) that indicates variability in particle size is not particularly limited. It may be 20% or less, preferably between 5% and 15%.

The average particle diameter is obtained by taking electronic microscope pictures with a scanning electron microscope (SEM), measuring cross sectional areas of a sufficient number of particles, and obtaining a diameter of a circle having an area of each measured value as a particle diameter. In the present application, the average particle diameter is a calculated average of particle diameters from a thousand particles. The coefficient of variation is also a value calculated from particle diameter distribution of a thousand particles.

(3) Antibody

An antibody (IgGs) which specifically recognizes an antigen of protein as the target biological material and which binds with the protein can be used as the primary antibody.

In the embodiment, an antibody that recognizes and binds with a particular domain of one target biologic material (protein) is used as the primary antibody. In addition, in the embodiment, antibodies that recognize different domains of one protein are selected as the primary antibodies that respectively bind with the two kinds of fluorescent substances.

For example, in a case in which PD-L1 is the target biological material, “SP263”, “SP142” (both made by Ventana), and “E1L3N” (made by Cell Signaling Technology) may be used as an anti-PD-L1 antibody that recognizes intracellular domains. Also, “22c3” (made by Dako) and “28-8” (made by Abcam) may be used as an anti-PD-L1 antibody that recognizes extracellular domains.

In a case in which HER2 is used as the target biological material, “4B5” (made by Ventana) and “CB11” (made by Bio Genex) are used as an anti-HER2 antibody that recognizes intracellular domains. Also, “SV2-61γ” (made by Nichirei Bioscience) may be used as an anti-HER2 antibody that recognizes extracellular domains.

In a case in which TIM-3 is used as the target biological material, “F38-2E2”, “RMT3-23” (both made by BioLegend), “MM0936-14S23” and “RM0135-6F46” (both made by Abcam) may be used as an anti-TIM-3 antibody that recognizes intracellular domains. Also, “344823” (made by R&D Systems) may be used as an anti-TIM-3 antibody that recognizes extracellular domains.

The primary antibody may be not a native full-length antibody but an antibody fragment or a derivative as long as it can specifically recognize and bind with a particular biological material (antigen). The term “antibody” herein includes not only a full-length antibody but also antibody fragments, such as Fab, F(ab)′2, Fv, scFv, and derivatives, such as a chimeric antibody (humanized antibody, etc.) and a multifunctional antibody.

As the secondary antibody, an antibody (IgG) that specifically recognizes the primary antibody as an antigen and binds therewith is used.

Both the primary antibody and the secondary antibody may be polyclonal antibodies; however, in view of stability of quantification, monoclonal antibodies are preferable. The type of an animal (immunized animal) which produces antibodies is not particularly limited, and a selection may be made from among mouse, rat, guinea pig, rabbit, goat, sheep, and the like similarly to traditional cases.

(4) Immunostainer

An immunostainer is made by dispersing a labeling antibody in a suitable medium. In the labeling antibody, an antibody that can directly or indirectly bind with the target biological material is directly or indirectly bound with a labeling substance.

To improve efficiency of fluorescent labeling and to prevent deterioration of fluorescence with the passage of time, it is preferable to use a complex in which the primary antibody and the phosphor integrated dots are indirectly connected with each other by a bond other than a covalent bond, that is, a bond utilizing antigen-antibody reaction or avidin-biotin reaction. However, others may be used.

The following is an example of an immunostainer indirectly combined with an antibody and fluorescent nanoparticles.

[primary antibody for target biological material] . . . [antibody for primary antibody (secondary antibody)]˜[fluorescent nanoparticle (phosphor integrated dot)]

The notation “. . . ” represents binding through an antigen-antibody reaction. The notation “˜” represents a bond which may be mediated by linker molecules according to necessity. The type of the bond is not limited and may be, for example, a covalent bond, an ionic bond, a hydrogen bond, a coordination bond, phsisorption, or chemisorption.

A conjugate of the secondary antibody˜phosphor integrated dot is made with, for example, a silane coupling agent which is a compound widely used for binding inorganic material and organic material. The silane coupling agent is a compound including an alkoxysilyl group providing a silanol group with hydrolysis in one end of the molecule and a functional group such as carboxy group, amino group, epoxy group, aldehyde group, and the like in the other end, and binds with the inorganic material through an oxygen atom of the silanol group. Specific examples include mercaptopropyl triethoxysilane, glycidoxypropyl triethoxysilane, aminopropyl triethoxysilane, silane coupling agent including polyethylene glycol chain (for example, PEG-silane no. SIM6492.7 manufactured by Gelest Inc.), and the like. In a case in which the silane coupling agent is used, two or more kinds may be used together.

Well-known methods can be used as the reaction method between the phosphor integrated dots and the silane coupling agent. For example, the obtained silica particles containing the fluorescent substance are dispersed in pure water, the aminopropyl triethoxysilane is added, and reaction is performed at room temperature for 12 hours. After the reaction ends, by centrifugal separation or filtration, it is possible to obtain silica particles containing the fluorescent substance and having a surface modified with the aminopropyl group. Next, the amino group is reacted with the carboxy group in the antibody so that the antibody binds with the silica particles containing the fluorescent substance through amide bond. If necessary, condensing agent such as EDC (1-Ethyl-3-[3-Dimethylaminopropyl] carbodiimide Hydrochloride: manufactured by Pierce) may also be used.

If necessary, a linker compound including a portion which can directly bind with the silica particles which contains the fluorescent substance and which is modified with the organic molecule, and a portion which can bind with the molecular target substance can be used. For example, when sulfo-SMCC (Sulfosuccinimidyl 4 [N-maleimidomethyl]-cyclohexane-1-carboxylate: manufactured by Pierce) which has a portion which selectively reacts with the amino group and a portion which selectively reacts with the mercapto group is used, the amino group of the silica particles which contains the fluorescent substance and which is modified with aminopropyl triethoxysilane and the mercapto group in the antibody are bound, and with this, the silica particles which contains the fluorescent substance and which is bound with the antibody is made.

When the biological material-recognizing portion (portion where a biological material is specifically recognized, such as biotin, avidin and an antibody) is bound with polystyrene particles containing the fluorescent substance, the same process can be applied no matter whether the fluorescent substance is the fluorescent organic dye or the quantum dot. In other words, by impregnating the fluorescent organic dye and the quantum dot in the polystyrene particles with the functional group such as the amino group, it is possible to obtain the phosphor integrated polystyrene particles with the functional group, and then by using the EDC or the sulfo-SMCC, the phosphor integrated polystyrene particles bound with the antibody is made.

Another example of an immunostainer indirectly combined with an antibody and fluorescent nanoparticles is a complex of three monocules combined as follows.

[primary antibody for target biological material] . . . [antibody for primary antibody (secondary antibody)]-[biotin]/[avidin]-[fluorescent substance (phosphor integrated dot)]

The notation “. . . ” represents binding through an antigen-antibody reaction. The hyphen “-” represents binding through a covalent bond which may be mediated by linker molecules according to necessity. The slash “/” represents binding through an avidin-biotin reaction.

A conjugate of the secondary antibody and biotin (biotin-modified secondary antibody) can be made in known methods for binding biotin with a desired antibody (protein). For example, biotin labeling reagents (kits) on the market may be used. If a biotin-modified secondary antibody in which biotin is already bound with a desired antibody is commercially available, it may be used.

A conjugate of the phosphor integrated dot and avidin (avidin-modified fluorescent substance) can also be made in known methods for binding avidin with a fluorescent substance. For example, avidin labeling reagents (kits) on the market may be used. In that case, avidin may be an improved avidin such as streptavidin or neutravidin. Bonding force is larger between the improved avidin and biotin than between avidin and biotin.

The following is an example of a method of preparing a conjugate of the phosphor integrated dot and avidin.

In a case in which the phosphor integrated dot includes a base of resin, a functional group of the resin is bound with a functional group of avidin (protein). The functional groups are bound via a linker molecule, such as PEG, having a functional group at both ends of the molecule if necessary. For example, in a case in which the base is a melamine resin, a functional group such as an amino group may be utilized. In a case in which the base is an acrylic resin, a styrene resin, etc., a monomer having a functional group (e.g., an epoxy group) on a side chain is copolymeried. Thereby the functional group itself or a functional group converted from the functional group (e.g., an amino group generated by reacting ammonia water) is utilized. Another functional group may be introduced utilizing those functional groups.

In a case in which the phosphor integrated dot includes a base of silica, a desired functional group is introduced by surface-modifying with a silane coupling agent. For example, an amino group is introduced by means of aminopropyltrimethoxysilane.

On the other hand, a thiol group is introduced into avidin by, for example, making N-succinimidyl S-acetylthioacetate (SATA) react with an amino group of avidin. Then, the avidin into which the thiol group is introduced is connected with the phosphor integrated dot having an amino group by means of a cross-linker reagent which includes N-hydroxysuccinimide (NHS) ester and a maleimide group at both ends of a polyethylene glycol (PEG) chain. N-hydroxysuccinimide (NHS) ester reacts with the amino group. The maleimide group reacts with the thiol group.

(5) Tissue Specimen

The tissue specimen refers to a tissue section collected from a subject (cancer patient), or cells obtained by culturing cells included in a tissue collected from a subject. In the present embodiment, a tissue section collected from a tumor tissue shall be used. The tissue specimen generally has the form of a specimen slide on which a tissue section or cells are placed, as commonly used in a case of assessing an expression level of a target protein through immunohistochemical staining or the like.

The method of making the tissue specimen is not particularly limited. The tissue specimen is generally obtained by, for example, cutting a tissue sample into sections of 3 to 4 μm, the tissue sample being made by fixing a tissue section collected from a subject using formalin or the like, performing xylene processing after dewatering with alcohol, immersing the tissue section in high-temperature paraffin to perform paraffin embedding. The tissue section is placed on a slide glass and dried to make a specimen slide.

(6) Staining Method

The staining method for the tissue specimen will be described. The staining method described below can be applied not only to the tissue section but also to cells.

(6.1) Making Specimen

### (6.1.1) Removing Paraffin

A tissue section is immersed in a container with xylene, and paraffin is removed. The temperature is not particularly limited, and the processing can be performed at room temperature. Preferably, the immersing time is between 3 minutes and 30 minutes. The xylene can be changed during the immersion if necessary.

Next, the tissue section is immersed in a container with ethanol, and the xylene is removed. The temperature is not particularly limited, and the processing can be performed at room temperature. Preferably, the immersing time is between 3 minutes and 30 minutes. The ethanol can be changed during the immersion if necessary.

The tissue section is immersed in a container with water to remove the ethanol. The temperature is not particularly limited, and the processing can be performed at room temperature. Preferably, the immersing time is between 3 minutes and 30 minutes. The water can be changed during the immersion if necessary.

### (6.1.2) Activating Processing

Activating processing of the target biological material is performed according to known methods. Activation condition is not particularly restricted, and examples of liquid for activation that can be used include, 0.01 M citric acid buffered solution (pH 6.0), 1 mM EDTA solution (pH 8.0), 5% urea, 0.1 M tris-hydrochloric acid buffered solution.

The pH is in a range between 2.0 and 13.0 and is determined in accordance with the tissue section so that a signal comes out and that the tissue is so rough that the signal can be assessed. Usually, the pH is between 6.0 and 8.0. In a case in which a special tissue section is used, the pH is 3.0, for example.

Examples of the heating device that can be used include autoclave, microwave, pressure pan, water bath, and the like. The temperature is not particularly limited, and the processing can be performed at room temperature. The processing can be performed at a temperature of 50 to 130° C. and the amount of time that the processing is performed can be 5 to 30 minutes.

After the activating processing, the tissue section is immersed in the container with PBS, and cleaning is performed. The temperature is not limited, and the processing can be performed at room temperature. Preferably, the immersing time is between 3 minutes and 30 minutes. The PBS can be changed during the immersion if necessary.

(6.2) Immunohistochemical Staining Step

In an immunohistochemical staining step, a solution of immunostainer is put on a tissue section and is made to react with a target biological material to stain the target biological material. The immunohistochemical staining step in the embodiment corresponds to the staining step in the information wiring method of the invention.

The solution of immunostainer used in the immunohistochemical staining step is prepared before this step.

Conditions for the immunohistochemical staining step, i.e., the temperature and an immersion time for immersing a tissue specimen in the solution of immunostainer, are adjusted in conventional immunohistochemical staining methods so that an appropriate signal is obtained.

The temperature is not particularly limited, and may be at room temperature. The reaction time is preferably between 30 minutes and 24 hours.

Before performing the above processing, it is preferable that a known blocking agent, such as BSA-containing PBS, and a surfactant, such as Tween20, are added dropwise.

For example, in a case in which the immunostainer is a complex of [primary antibody (probe)] . . . [secondary antibody]-[biotin]/[avidin]-[fluorescent nanoparticles (such as phosphor integrated dots)], preparation includes:

a first processing (primary reaction processing) of immersing a tissue specimen in a solution of the primary antibody;

a next processing (secondary reaction processing) of immersing the tissue specimen in a solution of the conjugate of the secondary antibody and biotin; and

a final processing (fluorescent labeling processing) of immersing a tissue section, which is the tissue specimen, in a solution of avidin-fluorescent nanoparticles dispersed in a diluent for the fluorescent nanoparticles according to the invention.

(6.3) Postprocessing for Specimen

The tissue specimen having undergone the immunohistochemical staining is preferably subjected to processing such as immobilization and dehydration, clearing, and sealing so as to be suitable for observation.

In the immobilization and dehydration processing, the tissue specimen may be immersed in a fixing processing liquid (a cross-linking agent such as formalin, paraformaldehyde, glutaraldehyde, acetone, ethanol, or methanol). In the clearing processing, the tissue specimen having undergone the immobilization and dehydration processing may be immersed in a clearing liquid (such as xylene). In the sealing processing, the tissue specimen having undergone the clearing processing may be immersed in a sealing liquid.

Conditions for performing these kinds of processing, the temperature and immersing time when immersing the tissue specimen in a predetermined processing liquid, for example, are adjusted as appropriate according to traditional immunostaining such that an appropriate signal is obtained.

(6.4) Form Observation Staining Step

In addition to the immunohistochemical staining step, form observation staining may be performed separately. Form observation staining allows observation of forms of cells, tissues, organs, etc. in the bright field.

Form observation staining step is performed in common methods.

Staining with eosin is generally performed to observe a form of a tissue specimen. Eosin stains cytoplasm, stroma, various fibers, erythrocytes, and keratinocytes in red or dark red. Staining with hematoxylin is also generally used. Hematoxylin stains cell nuclei, lime, cartilage tissue, bacteria, and mucus in a color ranging between indigo blue and light blue. A method of staining with both of them is known as hematoxylin and eosin staining (HE staining). Fluorescent dyes, such as DAPI (4′,6-diamidino-2-phenylindole), which specifically stains cellular nuclei, may be used for staining.

The form observation staining step may be performed after the immunohistochemical staining step and may be performed before the immunohistochemical staining step.

(7) Evaluation Method

### (7.1) Observation and Imaging Step

In an observation and imaging step, magnification of the micrograph acquiring device 1A is set at a desired value. A tissue specimen is illuminated with excited lights in one field of view. Excitation lights respectively correspond to fluorescent substances for fluorescent labeling on the target biological material used in the immunohistochemical staining step. Fluorescent images formed by fluorescence emitted from the fluorescent substances are observed and taken.

### (7.2) Quantification Step

In a quantification step, image processing of fluorescent images and quantification of expression levels are performed. The quantification step of the embodiment corresponds to a quantification step in the information acquiring method of the invention. The controller 21 as a quantification unit performs the quantification step.

Specifically, for fluorescent images taken with respect to the target biological material, the information acquiring device 2A measures fluorescent label signals, such as the number of fluorescent bright spots or fluorescent brightness, corresponding to the target biological material based on image processing. The information acquiring device 2A quantifies expression levels of the target biological materials in an area of cell membrane.

An example of software for image processing and quantification of expression levels is “ImageJ” (open source). Processing of extracting bright spots of a predetermined wavelength (color) from a fluorescent image, counting the number of bright spots of a predetermined brightness or higher, etc. is performed semi-automatically and rapidly by utilizing such an image processing software.

The bright spot comes from one of fluorescent nanoparticles. Therefore, size of the bright spot is constant, and the bright spots are recognized by microscopy. A signal larger than a constant value (e.g., an average value of observed fluorescent nanoparticles) is determined as an aggregated bright spot. The bright spot and the aggregated bright spot are semi-automatically and rapidly distinguished by a software.

### (7.3) Calculation Step

In a calculation step, a ratio between expression levels of domains, which are quantified in the quantification step, is calculated. Specifically, the ratio between expression levels is calculated from the number of bright spots in each target biological material, which is calculated in the quantification step. The calculation step in the embodiment corresponds to a calculation step in the information acquiring method of the invention. The controller 21 as a calculator performs the calculation step.

Like the above step, a software such as “ImageJ” may be used to calculate the ratio between expression levels of domains.

### (7.4) Evaluation Support Information Generation Step

In an evaluation support information generation step, evaluation support information is generated based on the ratio of expression levels of domains, which is calculated in the calculation step. The evaluation support information is useful for, for example, predicting prognoses of patients who provided tissue specimens. The generation step in the embodiment corresponds to a generation step in the information acquiring method according to the invention.

### EXAMPLES

Examples of the present invention will be described in detail, but the present invention is not limited thereto.

In this example, double staining of PD-L1, which is the targeted biological material, is performed with two kinds of fluorescent nanoparticles, that is, red phosphor integrated dots and green phosphor integrated dots.

(1) Immunostainer

In this example, one tissue specimen is stained with two kinds of immunostainers. One immunostainer uses the red phosphor integrated dots as a fluorescent label, and the other immunostainer uses the green phosphor integrated dots as a fluorescent label.

(1.1) ANTIBODY

An anti-PD-L1 rabbit monoclonal antibody “SP263”, which recognizes intracellular domains of PD-L1 tumor, was used as the primary antibody that indirectly binds with the red phosphor integrated dots. An anti-rabbit IgG antibody was used as the secondary antibody.

An anti-PD-L1 mouse monoclonal antibody “22c3”, which recognizes extracellular domains of PD-L1 tumor, was used as an antibody that binds with the green phosphor integrated dots. An anti-mouse IgG antibody was used as the secondary antibody.

(1.2) Synthesizing Red Phosphor Integrated Dots

### (1.2.1) Preparation of Biotin-Modified Anti-Rabbit IgG Antibody

A biotin-modified anti-rabbit IgG antibody was prepared through the following steps (I) to (IV).

### Step (I)

50 μg of the anti-rabbit IgG antibody as the secondary antibody was dissolved in a 50 mM Tris solution. A DTT (dithiothreitol) solution was added to this solution and mixed so that the final concentration became 3 mM. The reaction time was 30 minutes and the temperature was 37° C. Then, the reaction solution was passed through a desalting column “Zeba Desalt Spin Columns” (Thermo Scientific Inc., Cat. #89882). The secondary antibody reduced with DTT was purified. 200 μL of the purified antibody was dissolved in a 50 mM Tris solution to prepare antibody solution.

### Step (II)

A linker reagent “Maleimide-PEG2-Biotin” (Thermo Scientific Inc., Product Number 21901) was adjusted to be 0.4 mM by means of DMSO.

### Step (III)

8.5 μL of the linker reagent solution obtained in STEP (II) was added to the antibody solution obtained in STEP (I) and mixed. The reaction time was 30 minutes and the temperature was 37° C. As a result, biotin was bound with the anti-rabbit IgG antibody via a PEG chain. This reaction solution was passed through a desalting column and purified.

### Step (IV)

Absorbance of the reaction solution desalted in STEP (III) at a wavelength of 300 nm was measured with a spectral altimeter (“F-7000” made by Hitachi). Thereby concentration of protein (biotin-modified secondary antibody) in the reaction solution was calculated. Concentration of the biotin-modified secondary antibody in the solution of biotin-modified secondary antibody was adjusted to 250 μg/mL by means of a 50 mM Tris solution.

(1.2.2) Preparation of Texas Red Integrated Melamine Resin Dots

Texas Red integrated melamine resin dots were prepared through the following steps (I) to (IV).

### Step (I)

2.5 mg of Texas Red dye molecule “Sulforhodamine 101” (made by Sigma Aldrich) was dissolved in 22.5 mL of pure water. Then a hot stirrer stirred the solution for 20 minutes while keeping the temperature of the solution at 70° C.

### Step (II)

1.5 g of melamine resin “Nicalac MX-035” (made by Nippon Carbide Industries Co., Inc.) was added to the solution stirred in Step (I). The solution was heated and stirred under the same condition for 5 minutes. 100 μL of formic acid was added to the solution after stirring. The solution was stirred for 20 minutes while the temperature of the solution was kept at 60° C. Then, the solution was left to be cooled until the temperature of the solution reached room temperature.

### Step (III)

The solution cooled in STEP (II) was distributed among tubes for centrifugation. Centrifugation was performed at 12,000 rpm for 20 minutes. The Texas Red integrated melamine resin dots contained in the solution as a mixture was precipitated. The supernatant was removed and the precipitated particles were washed with ethanol and water.

### Step (IV)

SEM observation was performed on a thousand of the nanoparticles obtained in STEP (III). An average particle size was measured as described above. As a result, an average particle diameter was 152 nm.

(1.2.3) Preparation of Streptavidin-Bonded Texas Red Integrated Melamine Resin Dots

Streptavidin-bonded Texas Red integrated melamine resin dots were prepared through the following steps (I) to (VII).

### Step (I)

0.1 mg of Texas Red integrated melamine resin dots were dispersed in 1.5 mL of EtOH. 2 μL of amine propyltrimethoxysilane LS-3150 (made by Shin-Etsu Chemical) was added. After 8 hours of reaction, surface amination processing was performed.

### Step (II)

Concentration of particles subjected to the surface amination processing in STEP (I) was adjusted to 3 nM by means of PBS (phosphate buffer saline) containing 2 mM EDTA (ethylenediaminetetraacetic acid). SM(PEG) (Thermo Scientific Inc., succinimidyl—[(N-maleimidopropionamido)-dodecaethyleneglycol]ester) was mixed into this solution so that the final concentration became 10 mM. The reaction time of the solution was 1 hour.

### Step (III)

The mixture obtained in STEP (II) was subjected to centrifugation at 10,000 G for 20 minutes. After the supernatant was removed, PBS containing 2 mM EDTA was added. The sediment was dispersed and centrifugation was performed again. Washing in the same procedure was performed three times. Thus, phosphor integrated melamine dots having a maleimide group at its terminal was obtained.

### Step (IV)

Thiol group addition processing using N-succinimidyl S-acetylthioacetate (SATA) was performed on streptavidin (made by Wako Pure Chemical). Then, filtration was performed with a gel filtration column. Thus, a streptavidin solution that can bind with the phosphor integrated melamine dots was obtained.

### Step (V)

The phosphor integrated melamine dots obtained in STEP (III) and the streptavidin obtained in STEP (IV) were mixed in PBS that contains 2 mM EDTA. The reaction time was 1 hour and the temperature was room temperature.

### Step (VI)

10 mM mercaptoethanol was added to the mixture in STEP (V) to stop the reaction.

### Step (VII)

The solution obtained in STEP (VI) was concentrated with a centrifugal filter. Then, unreacted streptavidin, etc. was removed with a gel filtration column for purification. Thus, streptavidin-bonded phosphor integrated melamine dots were prepared.

(1.3) Synthesizing Green Phosphor Integrated Dots

### (1.3.1) Preparation of FITC-Modified Anti-Mouse Igg Antibody

A FITC-modified anti-mouse IgG antibody was prepared in the same procedure as “(1.2.1) Preparation of Biotin-Modified Anti-Rabbit IgG Antibody”. FITC was used instead of biotin.

### (1.3.2) Preparation of Alexa Fluor 488 Dye Integrated Melamine Resin Dots

Alexa Fluor 488 was used instead of the Texas Red dye molecule in “(1.2.2) Preparation of Texas Red Integrated Melamine Resin Dots”. Alexa Fluor 488 dye integrated melamine resin dots having an average particle size of 159 nm was prepared.

### (1.3.3) Preparation of Anti-FITC Antibody-Bonded Alexa Fluor 488 Integrated Melamine Resin Dots

An anti-FITC antibody was used instead of the streptavidin in “(1.2.3) Preparation of Streptavidin-Bonded Texas Red Integrated Melamine Resin Dots”. Thereby anti-FITC antibody-bonded Alexa Fluor 488 integrated melamine resin dots in which an antibody and fluorescent nanoparticles were directly bound.

(2) Staining Tissue Specimen

### (2.1) Preprocessing of Tissue Specimen

Tissue array slides “OD-CT-RsLug03-002” (hereinafter, referred to as Slide A) and “Hlug-Ade060PG-01” (hereinafter, referred to as Slide B) (both made by US Biomax) prepared with a lung cancer sample were used as tissue specimens.

As preprocessing for the tissue specimens, deparaffinization processing was performed on the tissue specimens, and washing was performed for replacement with water. Then, processing in an autoclave was performed on the washed tissue array slides in 10 mM citrate buffer (pH 6.0) for 15 minutes at 121° C. Thus, activating processing was performed on an antigen. After activating processing, tissue array slides were washed with PBS. Blocking processing was performed on the washed tissue array slides for 1 hour with PBS containing 1% BSA.

(2.2) Immunohistochemical Staining: Fluorescent Labeling with Red Phosphor Integrated Dots and Green Phosphor Integrated Dots

### (2.2.1) Primary Reaction Processing of Immunohistochemical Staining

In a primary reaction processing for the first immunostaining of the target biological material PD-L1, a primary reaction processing solution was prepared with PBS containing 1 W/W % BSA. Concentration of each of the anti-PD-L1 rabbit monoclonal antibody “SP263” and the anti-PD-L1 mouse monoclonal antibody “22c3” contained in the primary reaction processing solution was adjusted to be 0.05 nM. The specimens prepared in the specimen preprocessing step were immersed in the primary reaction processing solution. The temperature was 4° C., and the reaction time was one night.

### (2.2.2) Secondary Reaction Processing of Immunohistochemical Staining

A secondary reaction processing solution was prepared from each of:

the solution of biotin-modified anti-rabbit IgG antibody prepared in “(1.2.1) Preparation of Biotin-Modified Anti-Rabbit IgG Antibody”; and

the solution of FITC-modified anti-mouse IgG antibody prepared in “(1.3.1) Preparation of FITC-Modified Anti-Mouse IgG Antibody”.

Each solution was diluted with PBS containing 1 W/W % BSA so that the concentration became 6 μg/mL. After the primary reaction processing, the tissue specimen was washed with PBS and then immersed in the secondary reaction processing liquid. The reaction time was 30 minutes, and the temperature was room temperature.

(2.2.3) Fluorescent Labeling Processing: Labeling with Red Phosphor Integrated Dots and Green Phosphor Integrated Dots

A fluorescent label reaction processing solution was prepared from each of:

the streptavidin-bonded Texas Red integrated melamine resin dots prepared in “(1.2.3) Streptavidin-Bonded Texas Red Integrated Melamine Resin Dots Preparation”; and

the anti-FITC antibody-bonded Alexa Fluor 488 integrated melamine resin dots prepared in “(1.3.3) Preparation Of Anti-FITC Antibody-Bonded Alexa Fluor 488 Integrated Melamine Resin Dots”.

Each of the melamine resin dots was diluted with a dilution for fluorescent nanoparticles which contains 1% casein (composition: 50 W/W % α-casein (c6780 made by Sigma) and 50 W/W % β-casein (c6905 made by Sigma)) and 3% BSA so that the concentration of the fluorescent label reaction processing solution became 0.02 nM. After the secondary reaction processing, the specimens were immersed in the fluorescent label processing solution. The reaction time was 3 hours, and the temperature was room temperature.

(2.3) Form Observation Staining

For bright field observation, hematoxylin staining was performed by staining the specimens with Mayer's hematoxylin solution for 5 minutes. Then the specimens were washed with running water at 45° C. for 3 minutes. Eosin staining was then performed by staining with 1% eosin solution for 5 minutes.

(2.4) Post-Processing of Specimen

After the immunohistochemical staining, the tissue specimens were subjected to an immobilization and dehydration processing in which operation of immersing the tissue specimen in pure ethanol for 5 minutes was performed four times. Then, a thorough processing in which operation of immersing tissue specimen in xylene for 5 minutes was performed four times was performed. Finally, the encapsulant “Entellan new” (made by Merck) was put on the tissue specimen, and encapsulation processing in which a cover glass covers the tissue specimen was performed. Thus, specimens for observation were prepared.

(3) Evaluation Experiment

### (3.1) Observation and Photography

Immunohistochemical staining was performed on eighteen tissue specimens of Slides A and twelve tissue specimens of Slides B. The stained tissue specimens were illuminated with excitation light, and emitted fluorescence. Fluorescent images of the red phosphor integrated dots and the green phosphor integrated dots were acquired.

Fluorescence microscope “BX-53” (made by Olympus) was used for fluorescence observation. A digital camera “DP73” (made by Olympus) for microscope was attached to the fluorescent microscope and used to take fluorescent images.

First, the specimens were illuminated with excitation light corresponding to Texas Red dye, which was used as a fluorescent label of intracellular domains of the target biological material PD-L1, and emitted fluorescence. Thus, fluorescence images were acquired. The excitation wavelength was set between 575 nm and 600 nm by means of an optical filter for excitation light which was provided in the fluorescence microscope. The detection wavelength was set between 612 nm and 692 nm by an optical filter for fluorescence.

The specimens were then illuminated with excited light corresponding to the FITC dye used for fluorescent labeling of the extracellular domains of the target biological material PD-L1, and emitted fluorescence. Thus, fluorescence images were acquired. The excitation wavelength was set between 475 nm and 495 nm by means of an optical filter for excitation light which was provided in the fluorescence microscope. The detection wavelength was set between 510 nm and 534 nm by an optical filter for fluorescence.

The intensity of the excited light during observation and imaging by the fluorescent microscope was adjusted so that the irradiation energy around the center of the sight was 900 W/cm². The exposure time during imaging was adjusted so that the brightness of the images was not saturated, and was set at 4000 μs, for example.

(7.2) Measurement of the Number of Fluorescent Bright Spots and Calculation of Ratio

The expression level in the obtained fluorescence image in each color was specified by counting the number of fluorescent bright spots per cell, the fluorescent bright spots having a brightness equal to or higher than a predetermined value. The number of fluorescent bright spots in each color is an average of the number counted for approximately a thousand cells.

Further, a ratio of the number of green fluorescent bright spots to the number of red fluorescent bright spots per cell was calculated.

[Number of Green Fluorescent Bright Spots]/[Number of Red Fluorescent Bright Spots]=R

The software “ImageJ” was used for image processing.

Table 1 shows the number of fluorescent bright spots in each color and collected results of the value R.

“Sex” represents gender of a patient who provided a sample (M: male; F: female). “Grade” represents a degree of differentiation. “Stage” represents a stage of disease. “TNM” represents the TNM classification. “Survival Status” represents survival and decease. “Survival Months” represents a survival period (months) since sampling.

TABLE 1

| | | | | | | | | | Number of Particles per Cell | | |
|---|---|---|---|---|---|---|---|---|---|---|---|
| | | | | | | | Survival | Survival | Green Bright | Red Bright | |
| Slide | Sample | Sex | Age | Grade | Stage | TN M | Status | Months | Spots | Spots | R |
| Slide A | A1 | M | 53 | G1-G2 | IIA | T2bN 0M 0 | survival | 84 | 2.0 | 1.4 | 1.4 |
| | A2 | M | 77 | G1-G2 | II-III | T2aN xM 0 | deceased | 13 | 1.3 | 1.0 | 1.3 |
| | A3 | M | 57 | G1-G2 | IA | T1bN 0M 0 | survival | 87 | 1.9 | 1.2 | 1.7 |
| | A4 | M | 78 | G1-G2 | IA | T1bN 0M 0 | survival | 85 | 5.3 | 5.8 | 0.9 |
| | A5 | M | 75 | G2 | III | T4N xM 0 | deceased | 13 | 3.9 | 1.2 | 3.3 |
| | A6 | M | 61 | G2 | IB | T2aN 0M 0 | survival | 92 | 7.3 | 6.4 | 1.1 |
| | A7 | F | 70 | G2 | 0 | T1bN M 0 | deceased | 64 | 6.5 | 2.1 | 3.0 |
| | A8 | M | 58 | G2 | 0 | T2bN M 0 | survival | 90 | 2.6 | 1.2 | 2.2 |
| | A9 | M | 72 | G2 | IIA | T2bN 0M 0 | survival | 90 | 2.7 | 1.5 | 1.8 |
| | A10 | F | 59 | G2 | IIIA | T2aN 2M 0 | survival | 92 | 7.0 | 3.4 | 2.1 |
| | A11 | M | 69 | G2 | IA | T1bN 0M 0 | survival | 89 | 2.8 | 1.3 | 2.1 |
| | A12 | M | 61 | G2 | IIA | T2bN 0M 0 | survival | 87 | 10.7 | 2.4 | 4.5 |
| | A13 | M | 62 | G2 | IIA | T2aN 1M 0 | survival | 88 | 2.9 | 1.3 | 2.2 |
| | A14 | M | 70 | G2 | IA | T1bN 0M 0 | survival | 85 | 3.4 | 1.0 | 3.4 |
| | A15 | M | 61 | G2-G3 | IIIB | T3N 0M 0 | survival | 95 | 7.2 | 2.1 | 3.4 |
| | A16 | M | 63 | G2-G3 | IIIA | T4N 0M 0 | deceased | 58 | 3.6 | 1.7 | 2.1 |
| | A17 | F | 20 | G2-G3 | IIB | T2aN 0M 0 | survival | 83 | 3.9 | 3.4 | 1.1 |
| | A18 | M | 47 | G3 | IIA | T2aN 1M 0 | deceased | 20 | 2.0 | 2.2 | 0.9 |
| Slide B | B1 | F | 68 | G1 | IA | T1N 0M 0 | survival | 38 | 2.2 | 1.0 | 2.2 |
| | B2 | F | 58 | G1-G2 | IIB | T3N 0M 0 | survival | 56 | 3.5 | 1.7 | 2.1 |
| | B3 | F | 71 | G2 | II-III | T1N xM 0 | survival | 72 | 6.3 | 3.7 | 1.7 |
| | B4 | M | 51 | G2 | II-III | T1N xM 0 | survival | 70 | 4.1 | 1.5 | 2.8 |
| | B5 | M | 42 | G2 | II-III | T1N xM 0 | deceased | 13 | 4.3 | 1.7 | 2.5 |
| | B6 | M | 63 | G2 | II-III | T1N xM 0 | survival | 62 | 10.3 | 10.6 | 1.0 |
| | B7 | F | 60 | G2 | II-III | T1N xM 0 | survival | 57 | 2.0 | 1.0 | 2.1 |
| | B8 | F | 50 | G2 | II-III | T1N xM 0 | survival | 51 | 4.5 | 1.1 | 4. |
| | B9 | M | 40 | G2 | II-III | T1N xM 0 | survival | 44 | 3.8 | 1.8 | 2.1 |
| | B10 | F | 62 | G2 | II-III | T1N xM 0 | deceased | 10 | 7.1 | 2.7 | 2.7 |
| | B11 | F | 63 | G2-G3 | II-III | T2aN xM 0 | survival | 58 | 3.4 | 2.0 | 1.7 |
| | B12 | M | 67 | G3 | II-III | T2bN xM 0 | deceased | 39 | 21.4 | 5.9 | 3.6 |

As shown in Table 1, patients of samples with R less than 2.5 tend to have a longer survival period than patients of samples with R more than or equal to 2.5.

For example, Sample B6 (R=1.0) and Sample B10 (R=2.7) have the same degree of differentiation and the same stage. However, a survival period of a patient of Sample B6 is sixty two months, and that of a patient of Sample B12 is ten months. The survival period of the patient who provided Sample B6 is much longer.

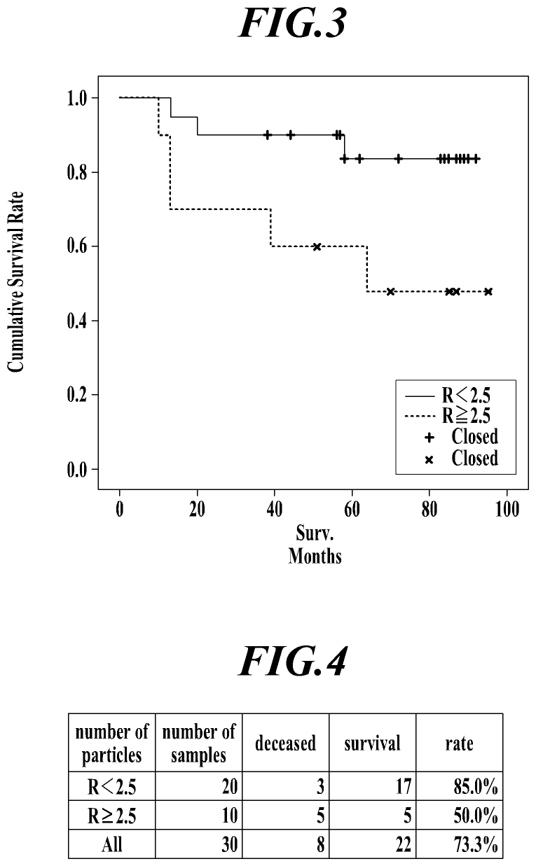

Statistical analysis as shown in FIG. 3 and FIG. 4 was performed to see correlation between R and the survival period.

FIG. 3 shows survival functions of patients who provided samples shown in Table 1. FIG. 4 shows survival rates of patients who provided samples. “Closed” in FIG. 3 includes decease of a patient who provided a sample. Significance probability “p” of the survival function is 0.042.

As is known from FIG. 3, the cumulative survival rate, i.e., the number of patients alive in the “survival months” shown on the horizontal axis, of samples with R less than 2.5 is larger than that of samples with R more than or equal to 2.5. The longer the survival months, the larger the difference in the cumulative survival rate.

As shown in FIG. 4, the final survival rate of samples with R more than or equal to 2.5 is 50% while the final survival rate of samples with R less than 2.5 is 85%. It is confirmed that patients who provided samples with R less than 2.5 is expected to have a longer survival period.

As described above, it turned out that there is some correlation between survival periods of patients and the value R, which is a ratio of the number of phosphor integrated dots that binds with extracellular domains of PD-L1 tumor to the number of phosphor integrated dots that binds with intracellular domains of PD-L1 tumor.

Thus, the example shows that quantitative relation of the expression level between the intracellular domains of PD-L1 tumor and the extracellular domains of PD-L1 tumor is one indicator for predicting prognoses of patients and can be utilized as evaluation support information.

In the embodiment, domains were stained with the phosphor integrated dots.

In the staining with a single fluorescent dye, fluorescence overlaps so that appearance of fluorescence changes. Therefore, it is difficult to clearly distinguish and separately stain domains. Also in dye staining, it is difficult to distinguish and separately stain domains because colors overlap. Contrary to them, the phosphor integrated dot has higher brightness and is smaller than a single fluorescent substance. Therefore, the phosphor integrated dots clearly distinguish and separately stain adjacent domains on one protein. As shown by the embodiment, the phosphor integrated dots are useful for staining for assessment by means of a ratio between expression levels of domains.

### Other Embodiments

Preferable embodiments of the present invention are explained above. The description in the above embodiments provides preferable examples of the present invention, but the present invention is not limited thereto.

Although the embodiment exemplifies the information acquiring method based on the numbers of bright spots of the phosphor integrated dots, the invention is not limited to this.

For example, intensity may be measured in an ELISA (Enzyme-linked immuno-sorbent assay) method. Specifically, fluorescent labeling with the phosphor integrated dots of different fluorescent wavelengths is performed respectively on different domains of one protein. Expression levels are quantified based on fluorescence intensity, and quantitative relation is evaluated. Alternatively, an enzymatic antibody method, such as DAB (3,3′-diaminobenzidine) method, may be used for labeling to quantify and evaluate expression levels.

### INDUSTRIAL APPLICABILITY

The present invention is suitable for providing an information acquiring method, an information acquiring device and a program that acquire information useful for diagnosis or treatment by quantitatively evaluating expression levels of domains of one protein.

### REFERENCE SIGNS LIST

100 Pathological diagnosis support system
1A Microscopic image acquiring device
2A Information acquiring device
21 Controller (quantification unit and calculator)
22 Operation interface
23 Display
24 Communicator I/F
25 Memory

1. An information acquiring method, comprising:
staining different domains of one protein in a tissue section of a human using staining reagents of different colors;
quantifying an expression level of each of the domains; and
calculating a ratio between expression levels of the domains.

2. The information acquiring method according to claim 1, wherein the tissue section is a sample from a tumor tissue of the human.

3. The information acquiring method according to claim 1, wherein the staining of the domains is performed in an immunohistochemical staining method.

4. The information acquiring method according to claim 3, wherein
each of the staining reagents is a combination of a biological material recognition site and phosphor integrated dots integrated with fluorescent substances;
the quantifying includes counting a number of bright spots of the phosphor integrated dots; and
in the calculating, a ratio between numbers of bright spots is calculated, the numbers being counted for the domains.

5. The information acquiring method according to claim 1, further comprising:
generating evaluation support information based on the ratio between the expression levels to predict prognosis of the human who provided the tissue section.

6. An information acquiring device that acquires information from a tissue section of a human by staining different domains of one protein in the tissue section using staining reagents of different colors, the device comprising:
a quantification unit that quantifies an expression level of each of the domains; and
a calculator that calculates a ratio between expression levels of the domains.

7. A non-transitory computer readable medium storing a program for a computer of an information acquiring device that acquires information from a tissue section of a human by staining different domains of one protein in the tissue section using staining reagents of different colors, the program making the computer function as:
a quantification unit that quantifies an expression level of each of the domains; and
a calculator that calculates a ratio between expression levels of the domains.
